(19) United States
(12) Patent Application Publication
(10) Pub. No.: US 2023/0018013 A1
(43) Pub. Date: Jan. 19, 2023

(54) Athlete Performance Tracking and Prediction System

(71) Applicant: PIFperformance Inc., Wilmington, DE (US)

(72) Inventor: David J. Allway, Ashburn, VA (US)

(21) Appl. No.: 17/841,224

(22) Filed: Jun. 15, 2022

Related U.S. Application Data
(60) Provisional application No. 63/339,589, filed on May 9, 2022.
(60) Provisional application No. 63/210,989, filed on Jun. 15, 2021.

(51) Int. Cl. A63B 24/00

(52) U.S. Cl. CPC A63B 24/0062; A63B 2024/0068

(57) ABSTRACT

An athlete performance tracking and prediction system may include a device processor and a non-transitory computer readable medium including instructions stored thereon, and executable by the processor, for performing the following steps: collecting data related to performance of a player into a database; compiling data and reporting performance attribute scores; performing comparisons between the reported performance attribute scores for the player to scores of other players in the database; and projecting future performance of the player based on the performed comparisons.

### CROSS-REFERENCE TO RELATED APPLICATIONS

This application claims priority to U.S. Provisional Patent Application No. 63/210,989, filed Jun. 15, 2021, and entitled “Athlete Performance Tracking and Prediction System,” and also claims priority to U.S. Provisional Patent Application No. 63/339,589, filed May 9, 2022, and entitled “Athlete Performance Tracking and Prediction System.” The entire disclosure of each application listed above is incorporated herein by reference.

### BACKGROUND

The present embodiments relate generally to athlete performance tracking systems and, in particular, to systems including performance prediction features.

Systems have been developed to track and predict athlete performance. In order to predict athlete performance, such systems consider a number of factors related to performance.

Existing systems are restricted to a predetermined set of performance factors considered by the performance prediction engine. That is, once the systems are deployed, the predictions made by the system will only be based on the factors that the system is configured to track at the time of deployment. However, new factors related to performance may be determined after the system is deployed.

There is a need in the art for a system and method that addresses the shortcomings discussed above.

### SUMMARY

In one aspect, the present disclosure is directed to an athlete performance tracking and prediction system, including a device processor and a non-transitory computer readable medium including instructions stored thereon, and executable by the processor, for performing the following steps: collecting data related to performance of a player into a database; compiling data and reporting performance attribute scores; performing comparisons between the reported performance attribute scores for the player to scores of other players in the database; and projecting future performance of the player based on the performed comparisons.

In another aspect, the present disclosure is directed to an athlete performance evaluation system. The system may include a device processor and a non-transitory computer readable medium including instructions stored thereon, and executable by the processor, for performing the following steps: collecting a first set of data related to a first performance metric from a first source; collecting a second set of data related to a second performance metric from a second source, wherein the second performance metric is substantially similar the first performance metric; converting the data related to the first performance metric to a new scale producing a third set of data; converting the data related to the second performance metric to the same new scale producing a fourth set of data; and comparing the third set of data to the fourth set of data.

In another aspect, the present disclosure is directed to an athlete performance evaluation system. The system may include a device processor and a non-transitory computer readable medium including instructions stored thereon, and executable by the processor, for performing the following steps: collecting a first set of data related to a first performance metric from a first source; collecting a second set of data related to a second performance metric from a second source, wherein the second performance metric is substantially similar the first performance metric; converting the data related to the first performance metric to the same scale as the second set of data producing a third set of data; and comparing the third set of data to the second set of data.

Other systems, methods, features, and advantages of the embodiments will be, or will become, apparent to one of ordinary skill in the art upon examination of the following figures and detailed description. It is intended that all such additional systems, methods, features, and advantages be included within this description and this summary, be within the scope of the embodiments, and be protected by the following claims.

### BRIEF DESCRIPTION OF THE DRAWINGS

The embodiments can be better understood with reference to the following drawings and description. The components in the figures are not necessarily to scale, emphasis instead being placed upon illustrating the principles of the embodiments. Moreover, in the figures, like reference numerals designate corresponding parts throughout the different views.

FIG. 1 is a venn diagram showing the overlap of various factors of the human performance model;

FIG. 2 is a table showing examples of measurements for the various factors of the human performance model;

FIG. 3 is a sample graph showing tracking of player performance relative to standards established based on other data;

FIG. 4 is a schematic diagram of a talent identification model;

FIG. 5 is a graph showing performance tracking of multiple players against one another and against predetermined standards;

FIG. 6 is an exemplary table of averaged player performance scores associated with FIG. 5;

FIG. 7 is an exemplary model of the cognitive processes involved in kicking a soccer ball and factors involved in evaluating this skill;

FIG. 8 is a table of a collection of tasks that may be all handled by the system;

FIG. 9 is a graphical representation of various tasks listed in FIG. 8 illustrating how they relate to one another;

FIG. 10 is a flowchart illustrating a method of projecting future performance;

FIG. 11 is a flowchart illustrating a method of converting similar metrics; and

FIG. 12 is a flowchart illustrating another method of converting similar metrics.

### DETAILED DESCRIPTION

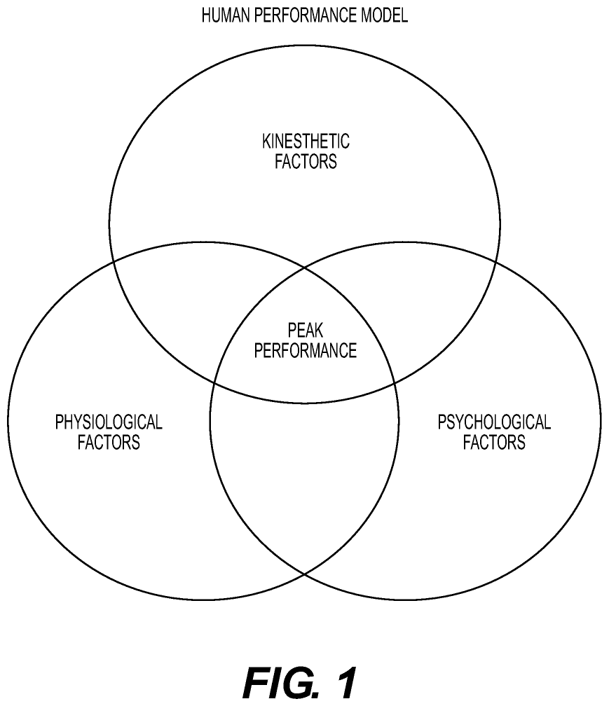

Human Performance is dictated by a combination of processes from three major areas. The first area involves physiological factors, such as a player's skill set, conditioning, and training. The second area involves psychological factors, such as a player's motivation, training, and readiness. The third area involves kinesthetic factors, such as a player's understanding of body location and positioning, and associated training. Combining these factors in the most effective way yields peak performance, as shown in FIG. 1. Conversely, human inefficiencies are a result of an individual's failure in one of these three key areas, i.e., physical ability, psychological readiness, and/or kinesthetic awareness.

The present disclosure is directed to a system configured to monitor and predict athlete performance based on these factors. The system may include a device processor and a non-transitory computer readable medium including instructions stored thereon that are executable by the device processor.

The non-transitory computer readable medium may include any suitable computer readable medium, such as a memory, e.g., RAM, ROM, flash memory, or any other type of memory known in the art. In some embodiments, the non-transitory computer readable medium may include, for example, an electronic storage device, a magnetic storage device, an optical storage device, an electromagnetic storage device, a semiconductor storage device, or any suitable combination of such devices. More specific examples of the non-transitory computer readable medium may include a portable computer diskette, a floppy disk, a hard disk, a read-only memory (ROM), a random access memory (RAM), a static random access memory (SRAM), a portable compact disc read-only memory (CD-ROM), an erasable programmable read-only memory (EPROM or Flash memory), a digital versatile disk (DVD), a memory stick, and any suitable combination of these exemplary media. A non-transitory computer readable medium, as used herein, is not to be construed as being transitory signals, such as radio waves or other freely propagating electromagnetic waves, electromagnetic waves propagating through a waveguide or other transmission media (e.g., light pulses passing through a fiber-optic cable), or electrical signals transmitted through a wire.

Instructions stored on the non-transitory computer readable medium for carrying out operations of the present invention may be instruction-set-architecture (ISA) instructions, assembler instructions, machine instructions, machine dependent instructions, microcode, firmware instructions, configuration data for integrated circuitry, state-setting data, or source code or object code written in any of one or more programming languages, including an object oriented programming language such as Smalltalk, C++, or suitable language, and procedural programming languages, such as the “C” programming language or similar programming languages.

Aspects of the present disclosure are described in association with figures illustrating flowcharts and/or block diagrams of methods, apparatus (systems), and computing products. It will be understood that each block of the flowcharts and/or block diagrams can be implemented by computer readable instructions. The flowcharts and block diagrams in the figures illustrate the architecture, functionality, and operation of possible implementations of various disclosed embodiments. Accordingly, each block in the flowchart or block diagrams may represent a module, segment, or portion of instructions. In some implementations, the functions set forth in the figures and claims may occur in an alternative order than listed and/or illustrated.

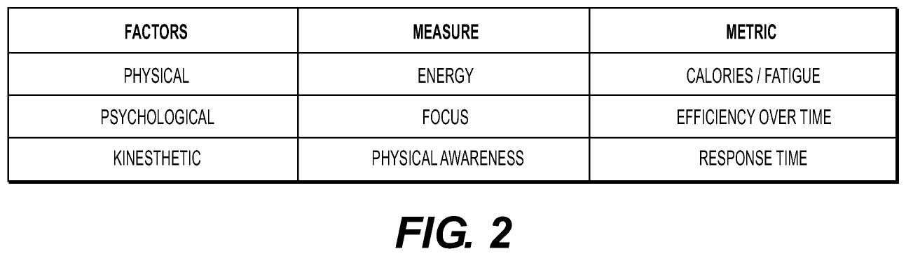

FIG. 2 is a table showing examples of measurements for the various factors of the human performance model. In particular, as shown in FIG. 2, in order to track a player's physical characteristics, energy can be measured using calories/fatigue as a metric. To track a player's psychological readiness, their focus can be measured using their efficiency over time as a metric. To track a player's kinesthetic awareness, the player's physical awareness may be measured using response time as a metric.

After data has been tracked across multiple players, standards may be established. For example, an upper limit and lower limit may be assessed within a particular organization, e.g., a team, club, league, etc. For example, the upper and lower limits may represent the performance of the best player and the worst player in the organization. Accordingly, if a player's performance tracks beyond the upper or lower limit, that may influence how that player is handled. For example, if the player's performance falls below the lower limit, that player may be released from the team. On the other hand, if the player's performance exceeds the upper limit representing the best of all players in the organization to that point, the staff will know that they have a special player on their hands and certain actions can be taken accordingly. In some cases, that may mean moving the player up an age group to provide a greater challenge. In a professional setting, it may mean selling off the player to a team in a stronger league.

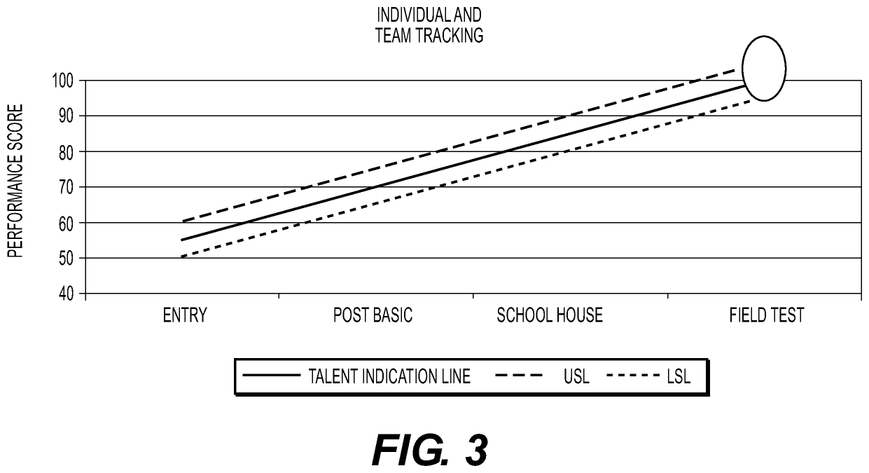

FIG. 3 is a sample graph showing tracking of player performance relative to standards established based on other data. As shown in FIG. 3, the upper limit is shown in a black dashed line and the lower limit is shown in a gray dashed line. The player in question is represented by the solid line that happens to fall between the limits in this case.

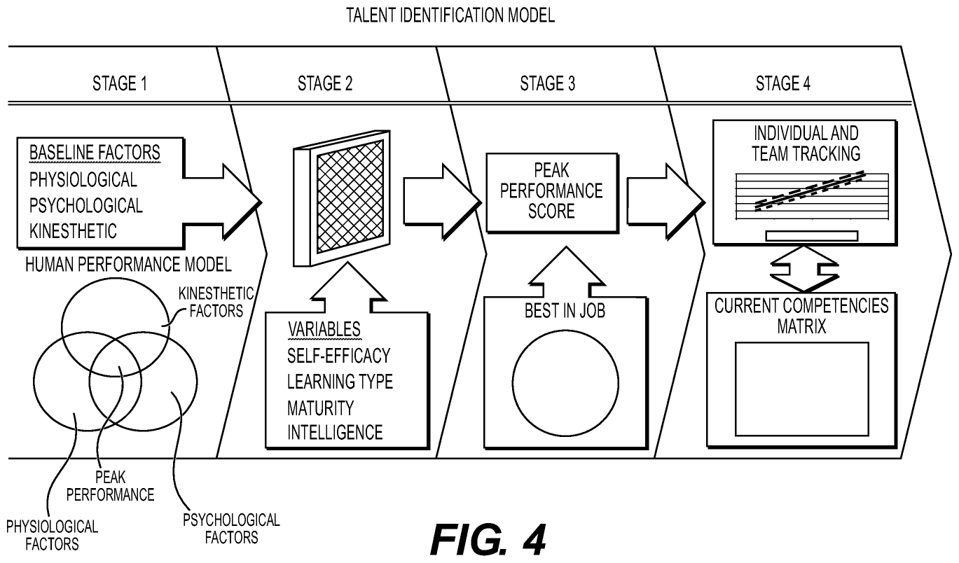

FIG. 4 is a schematic diagram of a talent identification model. As shown in FIG. 4, Stage 1 indicates the input of the baseline factors, in particular, physiological factors, psychological factors, and kinesthetic factors. Stage two represents the consideration of additional data, including several variables that affect performance. These variables include but are not limited to self-efficacy, learning type, maturity, and intelligence. This data may be filtered and analyzed to produce a peak performance score for the athlete at Stage 3. Further at Stage 3, the peak performance score may be compared to the best in job score (i.e., the upper limit discussed with respect to FIG. 3). In Stage 4, the performance of individuals and groups of individuals may be tracked over time and used to establish a matrix of current competencies. Moreover, these scores may be compared to the latest competencies to see where they fall.

To use an example, soccer players may be evaluated based on a series of attributes. An exemplary, albeit non-exhaustive, list of attributes that may be tracked in soccer players may include attendance, 1 v. 1 ability, 1 v. 1 ability to goal, 120 yard run performance, 120 yard shuttle runs, composite speed and agility testing, cones agility testing, cooper test distance, shooting, speed ladder, maturity, decision making, tactical awareness and implementation, motivation, creativity, awareness, self-efficacy, strength, flexibility, power, core values. These factors may be scored each month over the course of a year. The players' scores may be tallied for each month and averaged across the year. In addition, the scores may be weighted based on the players' attendance to practices and/or games. The monthly and/or yearly scores may be compared for a given player as well as player to player. In addition, different levels of administrative access enables different comparative analyses to be performed by different individuals, e.g., coaches, club administrators, league officials, scouts, etc.

Alternatively, or additionally, biomechanical metrics may be tracked, such as players' height, weight, body mass index, body proportions data (arm length, leg length, foot size, etc.), knee valgus angle, foot pronation/supination, and any other pertinent metrics. In some embodiments, the system may be configured to compare such metrics player to player. In some embodiments, the system may be configured to make performance predictions based on biomechanical metrics. In some cases, the system may be configured to predict youth players' physical growth based on the tracked biomechanical metrics. In some embodiments, the system may track and utilize biomechanical metrics and/or other performance metrics of players' parents in order to predict player physical growth and/or performance.

In some embodiments, injury data may be tracked. The system may be configured to predict injury risk based on injury data, biomechanical metrics, and/or player load. In addition, based on this injury risk and possibly based on other biomechanical metrics, the system may be configured to recommend programming to prevent injury. For example, a high knee valgus angle puts athletes at greater risk of injuries like anterior cruciate ligament (ACL) tears. Girls and women tend to have higher knee valgus angles, and thus can be more susceptible to ACL tears. In some embodiments, the system may be configured to recommend certain resistance exercises to strengthen the hips in order to provide stability for a player with a high knee valgus angle. The system may also be configured to recommend rest for players with high loading. In some embodiments, the system may be configured to recommend increased rest for players prior to and during puberty, when the players' bodies are not yet fully developed, and thus, at greater risk for injury. This timeline may be understood by the system by virtue of tracking the players' growth spurts and/or by comparing their biomechanical metrics with the players' parents. For example, if a player's parents are 6′0″ tall and 5′8″ tall, but the player is only 5′2″ tall, it can be assessed that the player has not completed their pubescent growth spurt. Until such a player approaches approximately 5′10″, for example, the system may recommend a reduced work load. Similarly, until the player completes the pubescent growth spurt, the system may recommend only body weight resistance training and not free weights.

It will also be noted that, in some embodiments, the player themselves may make inputs for some data. For example, players may be able to enter data related to their recruiting profile. For instance, the player may input certain data, like standardized testing scores that would be of interest to college scouts, but would not be otherwise available to the player's youth coach.

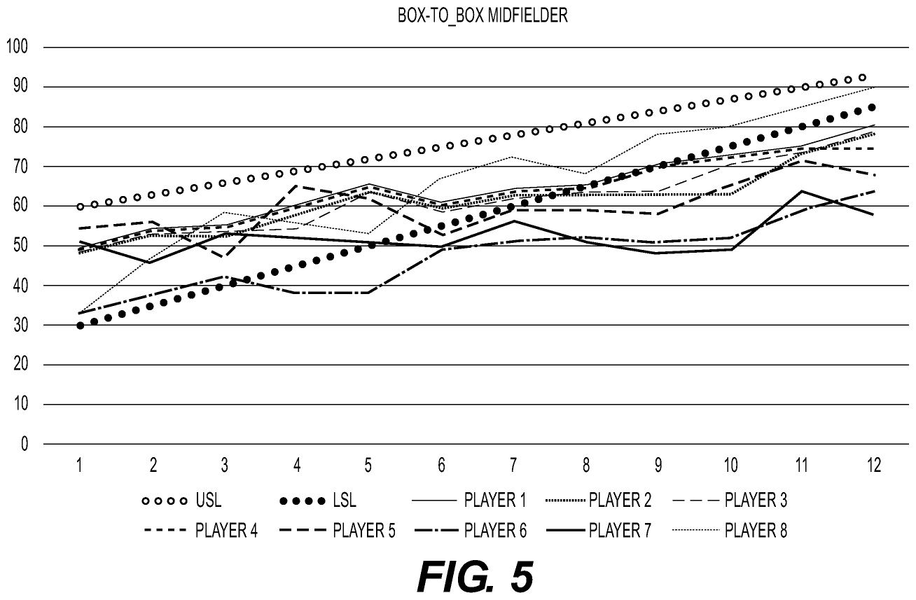

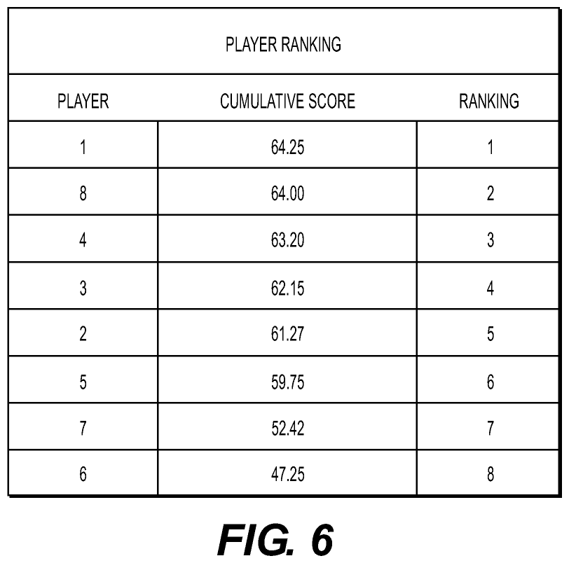

FIG. 5 is a graph showing performance tracking of multiple players against one another and against predetermined standards. In particular, FIG. 5 shows the progress of eight players over the course of 12 months. This graph specifically shows a collection of box to box midfielders. As shown, player 8 was the only one tracking within the upper and lower target limits by the end of the 12 month period. FIG. 6 is an exemplary table of averaged player performance scores associated with FIG. 5.

This data may be tracked over the course of weeks, months, years, etc. With knowledge of the path on which a player, or collection of players, is tracking, decisions can be made about interventions that can be implemented to improve the course of development. Such interventions may include the selection of practice topics, drills, game schedules, coaching staff, etc. to address areas where development may be lagging behind a predetermined standard or target.

In addition, while FIG. 5 shows the overall ratings for players, such metrics can be tracked and displayed for individual skills and attributes. For example, a similar graph may be generated for a player's ability to complete passes or for their fitness levels or for any other metric tracked by the system.

By tracking the various metrics, further evaluations may be made. For example, based on the player's profile, the system can recommend a position on the field that for which the player may be best suited. For instance, a player may be currently playing center midfield, which prioritizes skills such as physical endurance and passing accuracy. However, the player may be tracking strongest in metrics such as top speed and 1 v. 1 attacking. Accordingly, the system may suggest that the player is better suited for play as a winger.

In some embodiments, a player who leaves an organization may return to the organization after a hiatus and their data may be tracked with the same player profile that was used for them before they left. This enables for a better prediction of that player's future performance, as it considers data going back further than if the tracking merely started upon their return to the organization.

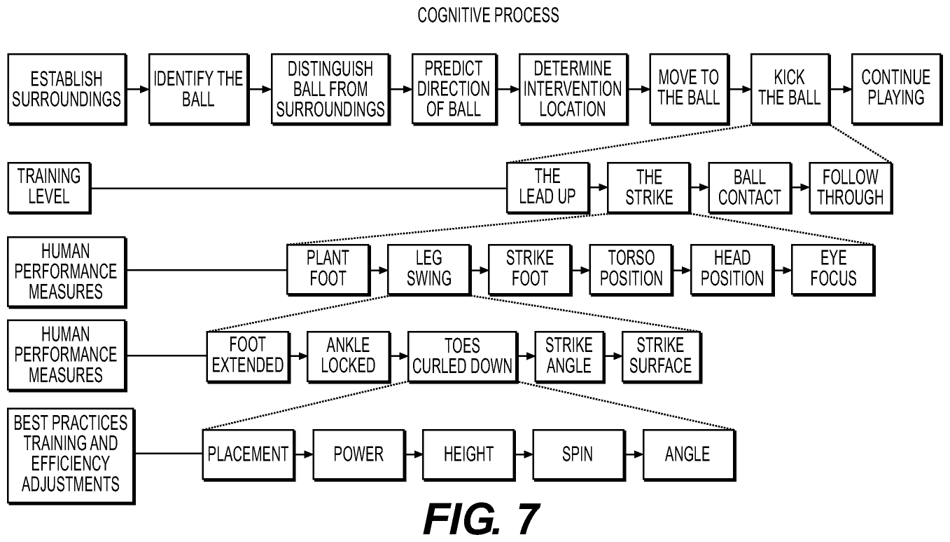

FIG. 7 is an exemplary model of the cognitive processes involved in kicking a soccer ball and factors involved in evaluating this skill. Aspects of each element on the chart shown in FIG. 7 may be monitored and tracked in order to evaluate a players performance with respect to kicking a ball.

The system may be configured to perform many tasks across several functional categories. Exemplary functional categories may include club management, team management, player development, and staff management. Examples of club management tasks may include training scheduling, game scheduling, player registration, player payments, document management, club communications. Examples of team management tasks may include rostering players, team brochures, game statistics, game attendance, training attendance, player ratings, player rankings, injury tracking, and team communications. Examples of player development tasks may include generating and maintaining player profiles, programming related to college recruiting education, hosting of a recruiting portal, linking to player highlight videos, reference management, lesson sharing and tracking, player evaluations, and player communications. Examples of staff management tasks may include lesson planning, lesson sharing, hosting a training video portal, player assessments, staff assessments, field assignments, game statistics, talent ID engine, and staff communications.

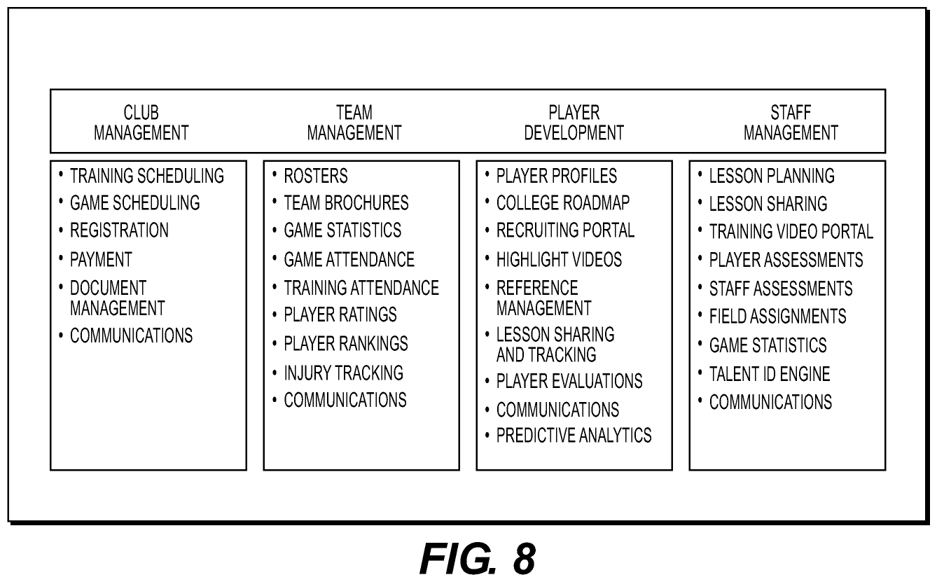

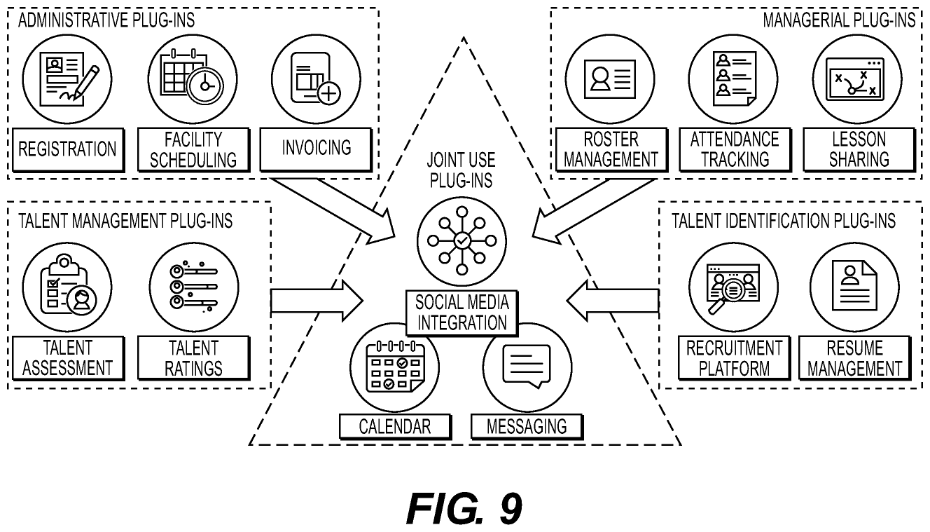

FIG. 8 is a table of a collection of tasks that may be all handled by the system. FIG. 9 is a graphical representation of various tasks listed in FIG. 8 illustrating how they relate to one another.

Player performance tracking and performance prediction is an ongoing and developing field. In some embodiments, the system may be configured to not only track player performance, but to also predict their future performance. However, because the field is continually developing, more different types of data becomes available. For example, GPS trackers have recently been developed to track players movement. In addition, performance research continues to reveal the importance of certain player performance attributes. Sometimes the attributes may be a metric for which statistics have not historically been kept. Accordingly, the collection of data regarding new metrics may be of significant interest. However, this presents a technological problem because player tracking systems are typically developed to track a predetermined set of attributes. However, in the present case, the system disclosed herein may be configured to enable consideration of new variables at any given time. That is, the system may be readily configurable to collect, monitor, compare new metrics, as well as make predictions and recommendations based on such data analysis.

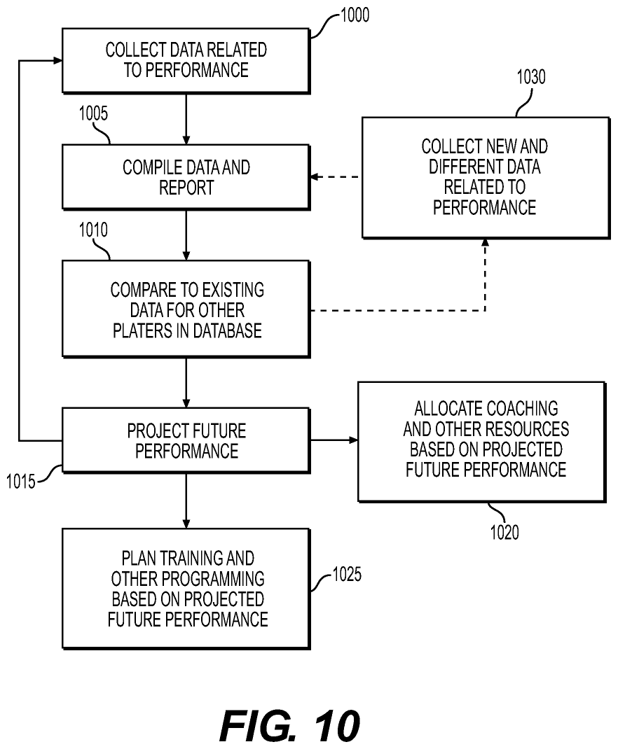

FIG. 10 is a flowchart illustrating a method of projecting future performance. To provide these predictive analytics, as shown in FIG. 10, data related to performance may be collected (step 1000). Then, the data may be compiled and one or more reports generated (step 1005). The reported player performance scores may then be compared to existing data for other players in the database (step 1010). In addition, the system may be configured to project future performance based on these comparisons (step 1015).

Further, based on the future performance predicted at step 1015, the system may offer suggestions for allocating coaching and other resources (step 1020). Alternatively or additionally, the system may be configured to plan training and other programming based on the projected future performance (step 1025).

The predictive capabilities of the system improve the more data is collected. That is, the more player data is collected, the more examples the system has as to what certain types of players go on to achieve. Therefore, as shown in FIG. 10, the system loops from the projections of future performance at step 1015 back to the collection of data at step 1000.

In addition, the system is flexible as to which variables are monitored to collect data. At any given time, the system may accept data regarding a new variable related to performance (step 1030). This enables the system to improve, taking advantage of advances in player tracking and evolution. For example, there are now electronic monitoring systems, both wearable and video based, that collect data regarding player performance. As these monitoring systems become more sophisticated, the new data they collect may be fed into the disclosed system, thus improving the data set upon which the future performance may be projected.

A technological problem is presented when trying to compare data across different organizations. In some cases, different organizations may track similar, but slightly different, metrics. In such cases, comparing data cannot be readily performed across organizations. This comes into play, for example, when a player is being evaluated for transfer from one club to another. A club considering buying a player from another club generally would prefer to compare the player's metrics to those of the purchasing club's own players.

In order to address this technological problem, another capability of the disclosed system is to translate ratings based on certain metrics into ratings for similar, but slightly different, metrics. For example, a given soccer club may collect data for players' 40-yard dash times, whereas another soccer club may collect data for players' 50-yard dash times. The times will be different, but both metrics measure essentially the same physical attributes, i.e., speed and acceleration. Another similar comparison is 40-yard dash times and 50-meter dash times, which are even more similar, yet still slightly different. The system may be configured to convert the data for one or both metrics so that the data is usable/meaningful to other organizations. That is, the data for players' 40-yard dash times may be meaningful and/or useful to the other club that typically records players' 50-yard dash times.

The system may do this in any of various ways. One exemplary way may be to convert both sets of data into a common ratings scale. For example, both sets of data may be converted to ratings on a scale of 10 or 100. Once all data entries are converted to the same scale, they can be compared club-to-club. This may facilitate player evaluation for purposes of college recruiting, professional drafts, player transfers, etc.

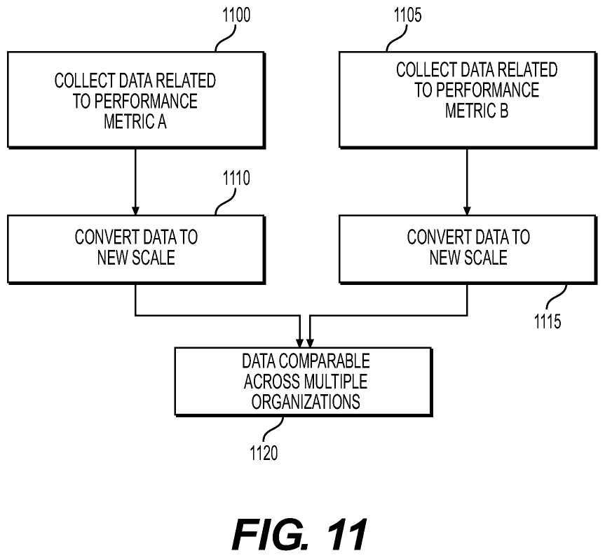

Such a method is illustrated in FIG. 11. As shown in FIG. 11, at step 1100, data relating to a first performance metric A may be collected. Similarly, at step 1105, data relating to a second performance metric B may be collected. It will be understood that these sets of data need not necessarily be collected in any particular order with respect to one another.

In addition, the data collected at step 1100 may be converted to a new scale at step 1110. Similarly, the data collected at step 1105 may be converted to a new scale at step 1115, wherein the new scale is common to both conversion processes. That is, both sets of data are converted to the same scale. Accordingly, at a further step 1120, the data may then be compared across multiple organizations, including both the organization that collected the data for metric A and the organization that collected the data for metric B.

Another exemplary way may be to convert one set of data into values that align with the other set of data. For example, the system may be configured to take 50-yard dash times and convert them to projected 40-yard dash times. This way the sprint performances recorded by the 50-yard dash club may be converted to times that are readily comparable to 40-yard dash times recorded by the other club. In some embodiments, the system may be configured to convert to the more commonly used metric. For example, 40-yard dash testing may be the more commonly used metric, and thus, the system may convert all similar sprint times to 40-yard dash times for comparison. In some embodiments, the system may be configured to convert in both directions, e.g., from 40 to 50 AND from 50 to 40. This way both clubs may readily evaluate the performance of players at the other club.

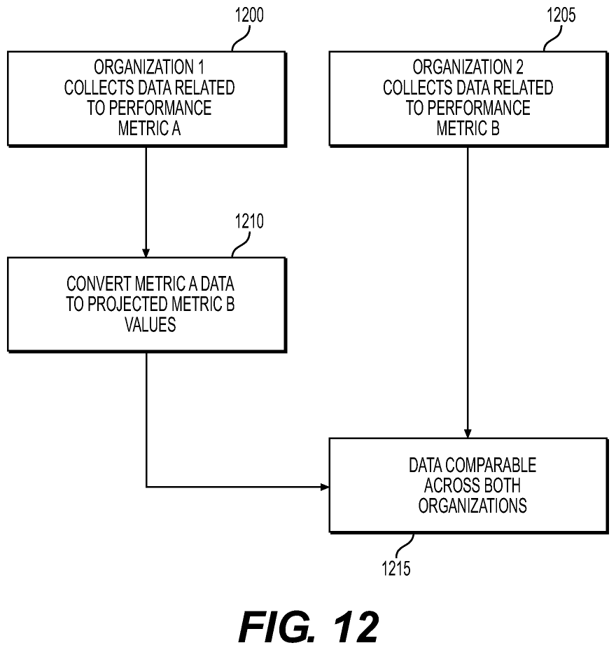

FIG. 12 illustrates the conversion method described above. In particular, at step 1200, a first organization may collect data related to a first performance metric A. At step 1205, a second organization may collect data related to a second performance metric B. Again, these steps need not necessarily occur in a particular order with respect to one another.

At step 1210, the data for metric A may be converted by projecting the results as metric B values. Accordingly, at step 1215, both sets of data may be compared across both organizations.

The conversion and comparison of similar metrics may be done for any type of metric recorded. This may be done for objective statistics, such as fitness times, passing accuracy percentages, etc., as well as subjective statistics, such as decision making or motivation, which may be scored on a simple point scale by a coach (e.g., with ratings on a scale of 1 to 10).

In some cases, the system may be configured to convert multiple metrics into one or more other metrics. For example, perhaps one club may record ratings for decision making (e.g., on a scale of 1 to 10). While another club may not record ratings specifically for decision making, they may record other metrics that are collectively indicative of decision making. For example, the second club may record metrics such as pass completion percentage, scoring chances created, and shots on goal. Collectively, these three metrics provide an excellent picture of a player's decision making. Accordingly, the system may be configured to take the data for two or more of these metrics and convert it to a “decision making” rating.

By this conversion and comparison of similar metrics, the data recorded and/or stored in the disclosed system may provide a universally useful tool across many organizations, despite each organization possibly recording metrics that may be slightly different from those collected by other organizations. It will be noted that the converted data may be useful not only to the organizations that originally collected the data (e.g., those that actually recorded the players' sprint times), but also to third party organizations who are interested in evaluating the players of other organizations.

Sometimes those tracking player metrics can become buried in an avalanche of data. That is, with so many metrics tracked, a technological problem is created because the data becomes less useful instead of more useful. For example, if all metrics are weighted the same, the performance evaluation and/or predictions are likely to be less accurate. In order to address this technological problem, the disclosed system may implement a weighting system. Such a weighting system may utilize correlation coefficients.

In some embodiments, correlation coefficients may be used to auto-adjust the performance evaluations by weighting the scores of each input individually. That is, each input has its own correlation coefficient associated with it, which weights the individual input based on which inputs are best at predicting future performance. In other words, each input score is weighted based on its “correlation” with performance. Y=f(x*weighted scores via the correlation coefficient)

$$\begin{matrix} \\ \\ Correlation coefficient ‐\end{matrix} \qquad r = \frac{\sum ( x - \overline{x} ) ( y - \overline{y} )}{\sqrt{\sum {( x - \overline{x} )}^{2} {( y - \overline{y} )}^{2}}}$$

The correlation coefficients account for change in the correlation between the inputs and performance by being changeable themselves.

The embodiments discussed herein may make use of methods and systems in artificial intelligence to improve efficiency and effectiveness of the disclosed systems. As used herein, “artificial intelligence” may include any known methods in machine learning and related fields. As examples, artificial intelligence may include systems and methods used in deep learning and machine vision. In the case of the present system, the interpretation of voluminous player performance data in the database may be facilitated and improved with artificial intelligence, such as machine learning.

While various embodiments are described, the description is intended to be exemplary, rather than limiting and it will be apparent to those of ordinary skill in the art that many more embodiments and implementations are possible that are within the scope of the disclosed embodiments. Although many possible combinations of features are shown in the accompanying figures and discussed in this detailed description, many other combinations of the disclosed features are possible. Any feature or element of any embodiment may be used in combination with or substituted for any other feature or element in any other embodiment unless specifically restricted. Further, unless otherwise specified, any step in a method or function of a system may take place in any relative order in relation to any other step described herein.

What is claimed is:

1. An athlete performance tracking and prediction system, comprising:
a device processor and a non-transitory computer readable medium including instructions stored thereon, and executable by the processor, for performing the following steps:
collecting data related to performance of a player into a database;
compiling data and reporting performance scores;
performing comparisons between the reported performance attribute scores for the player to performance attribute scores of other players in the database;
projecting future performance of the player based on the performed comparisons.

2. The system of claim 1, wherein the computer readable medium further includes instructions for:
collecting new and different types of data not previously collected; and
make new projections of future performance for players based at least in part on the new and different types of data collected.

3. The system of claim 1, wherein the computer readable medium further includes instructions for recommending that the player be played in a different position based on the performed comparisons between the player's performance attribute scores and the performance attribute scores of the other players.

4. The system of claim 1, wherein the computer readable medium further includes instructions for weighting performance scores of different performance attributes differently.

5. The system of claim 4, wherein weighting performance scores includes utilizing a correlation coefficient that is established based on the relative influence each tracked performance attribute has on overall performance.

6. The system of claim 1, wherein the computer readable medium further includes instructions for tracking performance attributes of soccer players.

7. The system of claim 1, wherein the computer readable medium further includes instructions for:
collecting a first set of data related to a first performance metric from a first source;
collecting a second set of data related to a second performance metric from a second source, wherein the second performance metric is substantially similar the first performance metric;
converting the data related to the first performance metric to a new scale producing a third set of data;
converting the data related to the second performance metric to the same new scale producing a fourth set of data; and
comparing the third set of data to the fourth set of data.

8. The system of claim 1, wherein the computer readable medium further includes instructions for:
collecting a first set of data related to a first performance metric from a first source;
collecting a second set of data related to a second performance metric from a second source, wherein the second performance metric is substantially similar the first performance metric;
converting the data related to the first performance metric to the same scale as the second set of data producing a third set of data; and
comparing the third set of data to the second set of data.

9. An athlete performance evaluation system, comprising:
a device processor and a non-transitory computer readable medium including instructions stored thereon, and executable by the processor, for performing the following steps:
collecting a first set of data related to a first performance metric from a first source;
collecting a second set of data related to a second performance metric from a second source, wherein the second performance metric is substantially similar the first performance metric;
converting the data related to the first performance metric to a new scale producing a third set of data;
converting the data related to the second performance metric to the same new scale producing a fourth set of data; and
comparing the third set of data to the fourth set of data.

10. The system of claim 9, wherein the computer readable medium further includes instructions for:
collecting new and different types of data not previously collected; and
make new projections of future performance for players based at least in part on the new and different types of data collected.

11. The system of claim 9, wherein the computer readable medium further includes instructions for recommending that the player be played in a different position based on the performed comparisons between the player's collected performance metrics data and the performance metric data of the other players.

12. The system of claim 9, wherein the computer readable medium further includes instructions for weighting performance metrics data of different performance attributes differently.

13. The system of claim 12, wherein weighting performance metrics data includes utilizing a correlation coefficient that is established based on the relative influence each tracked performance metric has on overall performance.

14. The system of claim 9, wherein the computer readable medium further includes instructions for tracking performance metrics of soccer players.

15. An athlete performance evaluation system, comprising:
a device processor and a non-transitory computer readable medium including instructions stored thereon, and executable by the processor, for performing the following steps:
collecting a first set of data related to a first performance metric from a first source;
collecting a second set of data related to a second performance metric from a second source, wherein the second performance metric is substantially similar the first performance metric;
converting the data related to the first performance metric to the same scale as the second set of data producing a third set of data; and
comparing the third set of data to the second set of data.

16. The system of claim 15, wherein the computer readable medium further includes instructions for:
collecting new and different types of data not previously collected; and
make new projections of future performance for players based at least in part on the new and different types of data collected.

17. The system of claim 15, wherein the computer readable medium further includes instructions for recommending that the player be played in a different position based on the performed comparisons between the player's collected performance metrics data and the performance metric data of the other players.

18. The system of claim 15, wherein the computer readable medium further includes instructions for weighting performance metrics data of different performance attributes differently.

19. The system of claim 18, wherein weighting performance metrics data includes utilizing a correlation coefficient that is established based on the relative influence each tracked performance metric has on overall performance.

20. The system of claim 15, wherein the computer readable medium further includes instructions for tracking performance metrics of soccer players.
